(19) United States
(12) Patent Application Publication
(10) Pub. No.: US 2025/0228755 A1
(43) Pub. Date: Jul. 17, 2025

(54) ANHYDROUS COMPOSITION THAT STABILIZES POORLY SOLUBLE INGREDIENTS AND PROVIDES EXCELLENT FEELING OF USE

(71) Applicant: AMOREPACIFIC CORPORATION, Seoul (KR)

(72) Inventors: Sunhei RHEE, Yongin-si (KR); Jonghyub KIM, Yongin-si (KR); Dawoon AHN, Yongin-si (KR)

(21) Appl. No.: 19/016,824

(22) Filed: Jan. 10, 2025

(30) Foreign Application Priority Data
Jan. 12, 2024 (KR) 10-2024-0005685
Jan. 9, 2025 (KR) 10-2025-0003334

(51) Int. Cl. A61K 8/34; A61Q 5/02; A61Q 19/00

(52) U.S. Cl. CPC A61K 8/342; A61Q 5/02; A61Q 19/007; A61K 2800/31; A61K 2800/33; A61K 2800/592; A61K 2800/652

(57) ABSTRACT

The present disclosure relates to an anhydrous composition, and the composition according to one aspect of the present disclosure includes: a fatty alcohol; an emollient; a wax; and a poorly soluble substance, and has a viscosity of 300 cSt or more at 30° C., and can stabilize poorly soluble substances that provide effects to the human body to increase the moisture retention in the skin or hair, and can simultaneously provide softness and shine to the hair upon use, and exhibit excellent feeling of use without stickiness or residual feeling on the hands.

### CROSS REFERENCE TO RELATED APPLICATION

The present application claims priority to Korean Patent Application No. 10-2024-0005685 filed Jan. 12, 2024, the entire contents of which are hereby incorporated by this reference. Further, the present application claims priority to Korean Patent Application No. 10-2025-0003334 filed Jan. 9, 2025, the entire contents of which are hereby incorporated by this reference.

### BACKGROUND OF THE INVENTION

### Field of the Invention

The present disclosure relates to an anhydrous composition that stabilizes poorly soluble substances and has excellent feeling of use.

### Description of the Related Art

Ceramide is a representative component of skin and hair, and is capable of exerting a unique skin barrier function by preventing moisture loss from the human body and blocking the inflow of harmful substances from the outside. However, ceramide has a fatal disadvantage in that it aggregates in cosmetic formulations or separates and precipitates into needle-like crystals. Although these poorly soluble substances may exhibit excellent effects on the human body, such as on the skin and hair, there are many substances that are difficult to dissolve and/or stabilize when applied to cosmetic formulations, and the like, and therefore have substantially little effect on the human body. Therefore, there is a need for developing a composition capable of stably dissolving and/or dispersing poorly soluble substances in cosmetic formulations, and the like.

Meanwhile, oil-type cosmetics (for example, hair oil), which are anhydrous compositions, can instantly form a coating film on the skin or hair to provide softness and shine. However, this effect is mostly only temporary, and the oil's inherent slippery nature can leave a residual feeling on the hands, causing inconvenience to users. Therefore, there is a need for developing a composition which is effectively absorbed into the skin or hair while simultaneously exhibiting a refreshing feeling of use without leaving any residual feeling on the hands when used, and can impart softness or shine to the skin or hair.

### SUMMARY OF THE INVENTION

An object of one aspect of the present disclosure is to provide an anhydrous composition that stabilizes poorly soluble substances and has excellent feeling of use.

An anhydrous composition according to one aspect of the present disclosure includes a fatty alcohol, an emollient, a wax, and a poorly soluble substance, and has a viscosity of 300 cSt or more at 30° C.

The anhydrous composition according to one aspect of the present disclosure can stabilize poorly soluble substances that provide effects to the human body in the formulation. The anhydrous composition according to one aspect of the present disclosure can stabilize poorly soluble substances that provide effects to the human body to increase the efficiency of delivery to the body, and as a result, can increase the moisture retention in the skin or hair, and can simultaneously provide softness and shine to the skin or hair and provide excellent feeling of use without stickiness or residual feeling.

### BRIEF DESCRIPTION OF THE DRAWINGS

FIG. 1 illustrates the results of confirming whether a poorly soluble substance is stably dissolved and dispersed in the formulations of an example (a high-viscosity oil formulation containing ceramide) according to one aspect of the present disclosure and a comparative example (a low-viscosity oil formulation containing ceramide).

FIG. 2 illustrates the results of an analysis using a LumiSizer to determine whether a poorly soluble substance is stably dissolved and dispersed in the formulations of the examples and comparative examples according to one aspect of the present disclosure.

FIG. 3 illustrates the results of analyzing the penetration of ceramide into hair using a Raman microscope. The left side of FIG. 3 is a cross-section of hair from an untreated group (control), and the right side thereof is a cross-section of hair treated with an example composition of the present invention (a high-viscosity oil formulation containing ceramide), with ceramide shown in red. It was observed that ceramide effectively penetrated into the cuticle layer of hair treated with an example composition of the present invention (a high-viscosity oil formulation containing ceramide).

### DETAILED DESCRIPTION OF THE INVENTION

It should be understood that various exemplary embodiments of the present document and the terms used therein are not intended to limit the technical features set forth herein to particular exemplary embodiments and include various changes, equivalents, or replacements for a corresponding exemplary embodiment.

The present inventors have developed an anhydrous composition that effectively dissolves and stabilizes poorly soluble substances, allowing them to exhibit their effects in the human body such as the skin or hair, and at the same time being less sticky and having excellent feeling of use.

One aspect of the present disclosure provides an anhydrous composition including: a fatty alcohol; an emollient; and a wax. One aspect of the present disclosure provides an anhydrous composition including: a fatty alcohol; an emollient; and a wax; and having a viscosity of 300 cSt or more at 30° C.

According to an embodiment, the anhydrous composition may have a viscosity at 30° C. in a range of 300 cSt or more, 350 cSt or more, 400 cSt or more, 450 cSt or more, 500 cSt or more, 550 cSt or more, 600 cSt or more, 650 cSt or more, 700 cSt or more, 750 cSt or more, 800 cSt or more, 850 cSt or more, 900 cSt or more, 950 cSt or more, 1,000 cSt or more, 10,000 cSt or less, 11,000 cSt or less, 12,000 cSt or less, 13,000 cSt or less, 14,000 cSt or less, 15,000 cSt or less, 20,000 cSt or less, 30,000 cSt or less, 40,000 cSt or less, 50,000 cSt or less, or a combination thereof (for example: 300 to 15,000 cSt), but the viscosity is not limited thereto. 30° C. may be the standard temperature for viscosity measurement. cSt may be a unit of measuring kinematic viscosity.

According to an embodiment of the present disclosure, when the viscosity of the anhydrous composition is equal to or more than the predetermined viscosity at 30° C., a poorly soluble substance (for example, ceramide, and the like) may be dissolved and uniformly distributed in the composition, the formulation is not separated, and stability is excellent. Meanwhile, when the viscosity of the anhydrous composition is less than the predetermined viscosity at 30° C., a poorly soluble substance such as ceramide is poor in formulation stability such as precipitation without being dissolved in the composition, or the separation of the formulation. In the composition according to one aspect of the present disclosure, the poorly soluble substance is uniformly distributed and stabilized, so that when the poorly soluble substance is applied to the human body, such as the skin or hair, the efficacy by the poorly soluble substance (for example, moisturizing power) may be effectively exhibited.

According to an embodiment, the anhydrous composition includes a fatty alcohol; an emollient; and a wax, and may have a viscosity in the above-described range, thereby effectively dissolving the poorly soluble substance and stabilizing the poorly soluble substance in the formulation.

According to an embodiment, the anhydrous composition may further include a poorly soluble substance.

One aspect of the present disclosure provides an anhydrous composition including: a fatty alcohol; an emollient; a wax; and a poorly soluble substance, and having a viscosity of 300 cSt or more at 30° C.

According to an embodiment, the anhydrous composition may have a viscosity at 30° C. in a range of 300 cSt or more, 350 cSt or more, 400 cSt or more, 450 cSt or more, 500 cSt or more, 550 cSt or more, 600 cSt or more, 650 cSt or more, 700 cSt or more, 750 cSt or more, 800 cSt or more, 850 cSt or more, 900 cSt or more, 950 cSt or more, 1,000 cSt or more, 10,000 cSt or less, 11,000 cSt or less, 12,000 cSt or less, 13,000 cSt or less, 14,000 cSt or less, 15,000 cSt or less, 20,000 cSt or less, 30,000 cSt or less, 40,000 cSt or less, 50,000 cSt or less, or a combination thereof (for example: 300 to 15,000 cSt), but the viscosity is not limited thereto. 30° C. may be the standard temperature for viscosity measurement. cSt may be a unit of measuring kinematic viscosity.

According to an embodiment, the fatty alcohol may be a C9-27 fatty alcohol, a C10-26 fatty alcohol, a C11-25 fatty alcohol, a C12-24 fatty alcohol, a C13-23 fatty alcohol, or a C14-22 fatty alcohol, but is not limited thereto. According to an embodiment, the fatty alcohol may be a saturated fatty alcohol. For example, the fatty alcohol may be a C14-22 saturated fatty alcohol. According to an embodiment, the fatty alcohol may be at least one selected from the group consisting of myristyl alcohol, cetyl alcohol, stearyl alcohol, and behenyl alcohol.

According to an embodiment, the fatty alcohol may be included in the composition in an amount of 0.01 to 10 wt %, but the amount is not limited thereto. For example, the fatty alcohol may be included in the composition in an amount of 0.01 wt % or more, 0.05 wt % or more, 0.1 wt % or more, 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, 10 wt % or more, 0.01 wt % or less, 0.05 wt % or less, 0.1 wt % or less, 0.5 wt % or less, 1 wt % or less, 1.5 wt % or less, 2 wt % or less, 2.5 wt % or less, 3 wt % or less, 3.5 wt % or less, 4 wt % or less, 4.5 wt % or less, 5 wt % or less, 5.5 wt % or less, 6 wt % or less, 6.5 wt % or less, 7 wt % or less, 7.5 wt % or less, 8 wt % or less, 8.5 wt % or less, 9 wt % or less, 9.5 wt % or less, 10 wt % or less, or a combination thereof.

The “emollient” is an oil-soluble substance that enhances shine, and may soften the feel of the skin or hair surface, or form a moisturizing film.

According to an embodiment, the emollient may be at least one selected from the group consisting of an ester-based emollient, a triglyceride-based emollient, and a hydrocarbon-based emollient.

According to an embodiment, the ester-based emollient may be an ester of a carboxylic acid and a C3-C20 alcohol. According to an embodiment, the ester-based emollient may be an ester of a C3-C14 carboxylic acid and a C3-C20 alcohol, an ester of a C3-C14 carboxylic acid and a C9-C20 alcohol, an ester of a C4-C13 carboxylic acid and a C10-C19 alcohol, an ester of a C5-C12 carboxylic acid and a C10-C18 alcohol, an ester of a C6-C11 carboxylic acid and a C11-C17 alcohol, or the ester-based emollient may be an ester of a C7-C10 carboxylic acid and a C12-C16 alcohol, but is not limited thereto. According to an embodiment, the ester-based emollient may be at least one selected from the group consisting of cetyl ethylhexanoate, a C12-15 alkyl benzoate, and isopropyl myristate.

According to an embodiment, the triglyceride-based emollient may be esters of a C3-C22 carboxylic acid and glycerol, a C4-C14 carboxylic acid and glycerol, a C5-C13 carboxylic acid and glycerol, a C6-C12 carboxylic acid and glycerol, a C7-C11 carboxylic acid and glycerol, and a C8-C10 carboxylic acid and glycerol, but is not limited thereto. According to an embodiment, the triglyceride-based emollient may be at least one selected from the group consisting of caprylic/capric triglyceride, camellia oil, and Abyssinian oil.

According to an embodiment, the hydrocarbon-based emollient may be at least one selected from the group consisting of hydrogenated polyisobutene, squalane, squalene, and polybutene.

According to an embodiment, the emollient may be included in the composition in an amount of 0.1 to 15 wt %, but the amount is not limited thereto. For example, the emollient may be included in the composition in an amount of 0.1 wt % or more, 0.5 wt % or more, 1 wt % or more, 2 wt % or more, 3 wt % or more, 4 wt % or more, 5 wt % or more, 6 wt % or more, 7 wt % or more, 8 wt % or more, 9 wt % or more, 10 wt % or more, 11 wt % or more, 12 wt % or more, 13 wt % or more, 14 wt % or more, 15 wt % or more, 0.1 wt % or less, 0.5 wt % or less, 1 wt % or less, 2 wt % or less, 3 wt % or less, 4 wt % or less, 5 wt % or less, 6 wt % or less, 7 wt % or less, 8 wt % or less, 9 wt % or less, 10 wt % or less, 11 wt % or less, 12 wt % or less, 13 wt % or less, 14 wt % or less, 15 wt % or less, or a combination thereof.

According to an embodiment, the wax may be natural wax, but is not limited thereto. According to an embodiment, the wax may be at least one selected from the group consisting of jojoba esters, MANGIFERA INDICA (MANGO) seed butter, ASTROCARYUM MURUMURU seed butter, and hydrogenated vegetable oil, but is not limited thereto.

According to an embodiment, the wax may have a melting point of 25° C. to 75° C. According to an embodiment, the wax may have a melting point of 25° C. or more, 26° C. or more, 27° C. or more, 28° C. or more, 29° C. or more, 30° C. or more, 31° C. or more, 32° C. or more, 33° C. or more, 34° C. or more, 35° C. or more, 36° C. or more, 37° C. or more, 38° C. or more, 39° C. or more, 40° C. or more, 41° C. or more, 42° C. or more, 43° C. or more, 44° C. or more, 45° C. or more, 46° C. or more, 47° C. or more, 48° C. or more, 49° C. or more, 50° C. or more, 51° C. or more, 52° C. or more, 53° C. or more, 54° C. or more, 55° C. or more, 56° C. or more, 57° C. or more, 58° C. or more, 59° C. or more, 60° C. or more, 61° C. or more, 62° C. or more, 63° C. or more, 64° C. or more, 65° C. or more, 66° C. or more, 67° C. or more, 68° C. or more, 69° C. or more, 70° C. or more, 71° C. or more, 72° C. or more, 73° C. or more, 74° C. or more, 75° C. or more, 25° C. or less, 26° C. or less, 27° C. or less, 28° C. or less, 29° C. or less, 30° C. or less, 31° C. or less, 32° C. or less, 33° C. or less, 34° C. or less, 35° C. or less, 36° C. or less, 37° C. or less, 38° C. or less, 39° C. or less, 40° C. or less, 41° C. or less, 42° C. or less, 43° C. or less, 44° C. or less, 45° C. or less, 46° C. or less, 47° C. or less, 48° C. or less, 49° C. or less, 50° C. or less, 51° C. or less, 52° C. or less, 53° C. or less, 54° C. or less, 55° C. or less, 56° C. or less, 57° C. or less, 58° C. or less, 59° C. or less, 60° C. or less, 61° C. or less, 62° C. or less, 63° C. or less, 64° C. or less, 65° C. or less, 66° C. or less, 67° C. or less, 68° C. or less, 69° C. or less, 70° C. or less, 71° C. or less, 72° C. or less, 73° C. or less, 74° C. or less, 75° C. or less, or a combination thereof, but the melting point is not limited thereto.

According to an embodiment, the wax may be included in the composition in an amount of 0.01 to 10 wt %, but the amount is not limited thereto. For example, the wax may be included in the composition in an amount of 0.01 wt % or more, 0.05 wt % or more, 0.1 wt % or more, 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, 10 wt % or more, 0.01 wt % or less, 0.05 wt % or less, 0.1 wt % or less, 0.5 wt % or less, 1 wt % or less, 1.5 wt % or less, 2 wt % or less, 2.5 wt % or less, 3 wt % or less, 3.5 wt % or less, 4 wt % or less, 4.5 wt % or less, 5 wt % or less, 5.5 wt % or less, 6 wt % or less, 6.5 wt % or less, 7 wt % or less, 7.5 wt % or less, 8 wt % or less, 8.5 wt % or less, 9 wt % or less, 9.5 wt % or less, 10 wt % or less, or a combination thereof.

The “poorly soluble substance” refers to a substance that is not sufficiently soluble in water or oil, is not sufficiently soluble, and easily precipitates and exist unstably in a formulation, and refers to a component that can be used in a composition to impart useful effects to the skin and/or hair, such as wrinkle amelioration, skin whitening, and skin moisturizing.

The composition according to one aspect of the present disclosure includes a fatty alcohol, an emollient, and a wax, has a viscosity in a predetermined range, and thus can stably dissolve poorly soluble substances, and can exhibit useful effects on the skin or hair.

According to an embodiment, the poorly soluble substance may be one or more selected from the group consisting of, for example, ceramide, madecassoside, hyaluronic acid, retinol, retinoid, coenzyme Q-10, ursolic acid, oleanolic acid, lipoic acid, proteins, and water-soluble substances, as a poorly soluble physiologically active substance.

According to an embodiment, ceramide is a molecule in which a fatty acid is bonded to a sphingoid base. In this case, the sphingoid base may be at least one of sphingosine, dihydroxysphingosine, phytosphingosine, and 6-hydroxysphingosine, and the fatty acid may be at least one of natural fatty acid, alpha-hydroxy fatty acid, ester-linked omega hydroxy fatty acid, omega hydroxy fatty acid, and 1-O-acrylamide. For example, the ceramide may be at least one selected from the group consisting of phytosphingosine ceramide, sphingosine ceramide, dihydrosphingosine ceramide, and 6-hydroxysphingosine ceramide, but is not limited thereto. For example, the ceramide may be at least one selected from the group consisting of ceramide NS, ceramide AS, ceramide EOS, ceramide NDS, ceramide ADS, ceramide EODS, ceramide NP, ceramide AP, ceramide EOP, ceramide NH, ceramide AH, and ceramide EOH.

According to an embodiment, the poorly soluble substance may be one or more selected from the group consisting of ceramide NS, ceramide AS, ceramide EOS, ceramide NDS, ceramide ADS, ceramide EODS, ceramide NP, ceramide AP, ceramide EOP, ceramide NH, ceramide AH, ceramide EOH, madecassoside, hyaluronic acid, retinol, retinoid, coenzyme Q-10, ursolic acid, oleanolic acid, lipoic acid, proteins, and water-soluble substances, but is not limited thereto.

According to an embodiment, the poorly soluble substance may be included in the composition in an amount of 0.1 to 1.5 wt %, but the amount is not limited thereto. For example, the poorly soluble substance may be included in the composition in an amount in a range of 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1 wt % or more, 1.1 wt % or more, 1.2 wt % or more, 1.3 wt % or more, 1.4 wt % or more, 1.5 wt % or more, 0.1 wt % or less, 0.2 wt % or less, 0.3 wt % or less, 0.4 wt % or less, 0.5 wt % or less, 0.6 wt % or less, 0.7 wt % or less, 0.8 wt % or less, 0.9 wt % or less, 1 wt % or less, 1.1 wt % or less, 1.2 wt % or less, 1.3 wt % or less, 1.4 wt % or less, 1.5 wt % or less, of a combination thereof.

According to an embodiment, the anhydrous composition may include at least one selected from the group consisting of dimethicone, dimethiconol, cyclomethicone, and oil as a base. The oil may be cetyl ethylhexanoate, a C12-15 alkyl benzoate, and a C9-12 alkane, but is not limited thereto.

In the present disclosure, the “base” may refer to a component that forms the basic matrix of a composition. The base serves to stably dissolve or disperse other active ingredients, and may affect the viscosity, feeling of use, stability, and the like of the composition.

The anhydrous composition according to one aspect of the present disclosure may further include at least one of dimethicone, dimethiconol, and oil, but is not limited thereto. Dimethicone, dimethiconol and/or oil may be components included in the base of the anhydrous composition according to one aspect of the present disclosure. According to an embodiment, the viscosity of the composition of the present disclosure may be adjusted by dimethicone and/or dimethiconol.

According to an embodiment, the fatty alcohol, emollient, and wax may form particles in the anhydrous composition. According to one aspect of the present disclosure, the fatty alcohol, emollient, and wax may be present in the form of particles in the anhydrous composition. The particles may be solid lipid particles. An anhydrous composition according to one aspect of the present disclosure includes particles including a fatty alcohol, an emollient, and a wax; and a poorly soluble substance; and may have a viscosity of 300 cSt or more at 30° C.

According to an embodiment, the anhydrous composition may not include a surfactant. According to an embodiment, the anhydrous composition of the present disclosure may effectively dissolve and/or disperse poorly soluble substances in the anhydrous formulation even without a surfactant, and may form a stable formulation.

According to an embodiment, the anhydrous formulation may be an oil formulation, specifically, an anhydrous oil formulation.

In an embodiment, the composition may be a cosmetic composition, a food composition, an oral composition, a non-therapeutic composition, a non-therapeutic oral composition, a pharmaceutical composition, or an external preparation composition. For example, the composition may be a cosmetic composition or an external preparation composition.

In an embodiment, the composition may further include an additive such as a preservative, a thickener, a viscosity modifier, a stabilizer, a pearlizing agent, a metal ion sequestrant, a cationic surfactant, a pH adjuster, a fragrance, and a dye, which are readily available commercially.

The anhydrous composition according to one aspect of the present disclosure may be for skin, nails, toenails, or hair.

According to an embodiment, the anhydrous composition may be at least one of a hair serum, a hair oil, a hair essence, and a hair cream, but is not limited thereto. According to an embodiment, the anhydrous composition may be at least one selected from the group consisting of skin lotion, cream, essence, hand cream, mist, and oil, but is not limited thereto.

The anhydrous composition according to one aspect of the present disclosure may be for enhancing body moisturization. In an embodiment, the body further includes at least one selected from the group consisting of hair (for example: scalp, eyelashes, and eyebrows), skin (for example: elbows and cuticles), nails, and toenails, but is not limited thereto.

The anhydrous composition according to one aspect of the present disclosure may stabilize poorly soluble substances that have effects on the human body, and effectively deliver the poorly soluble substances to enhance the moisturizing effect or moisturizing durability in the body (for example: skin, hair, nails, and toenails), and may simultaneously exhibit an excellent feeling of use upon use.

The anhydrous composition according to one aspect of the present disclosure may stabilize poorly soluble substances to enhance the moisturizing effect or moisturizing durability in the body (for example: skin, hair, nails, and toenails).

The anhydrous composition according to one aspect of the present disclosure may provide softness and/or shine to the body (for example: skin, hair, nails, and toenails) upon use.

The anhydrous composition according to one aspect of the present disclosure may exhibit excellent feeling of use without stickiness or residual feeling on the hands upon use.

The anhydrous composition according to one aspect of the present disclosure may be absorbed into the body (for example: skin, hair, nails, and toenails) without leaving any residual feeling on the hands upon use.

In an embodiment, the anhydrous composition (for example: a high-viscosity oil formulation) according to one aspect of the present disclosure was able to effectively stabilize a poorly soluble substance (for example: ceramide) and had excellent formulation stability compared to other formulation compositions (for example: low-viscosity oil formulations and low-viscosity emulsion formulations). Furthermore, the anhydrous composition (for example: a high-viscosity oil formulation) according to one aspect of the present disclosure had excellent efficiency in delivering a poorly soluble substance (for example: ceramide) into hair (for example: the cuticle layer of hair) compared to other formulation compositions (for example: low-viscosity oil formulations and low-viscosity emulsion formulations).

One aspect of the present disclosure provides a method for enhancing moisturization of the body, the method including: applying the above-described anhydrous composition to a subject. One aspect of the present disclosure provides a method for enhancing moisturization of the body, the method including: applying the above-described anhydrous composition to the body of a subject. The body further includes at least one selected from the group consisting of hair (for example: scalp, eyelashes, and eyebrows), skin (for example: elbows and cuticles), nails, and toenails, but is not limited thereto.

In addition, one aspect of the present disclosure provides a method for enhancing moisturization of hair, skin, nails, or toenails, the method including: applying the above-described anhydrous composition to a subject.

In an embodiment, the subject may be an animal including a mammal (for example: a human), but is not limited thereto.

In the present disclosure, application may be oral, transdermal, subcutaneous, intravenous, intraperitoneal, intramuscular, or topical, but is not limited thereto, and preferably, application may include applying the above-described anhydrous composition to the hair.

In an embodiment, the daily dosage of the anhydrous composition may be 0.0001 to 10,000 mg/kg, but is not limited thereto. In an embodiment, the anhydrous composition may be applied once or several times a day. However, it is to be understood that the dosage of the anhydrous composition needs to be determined in consideration of various related factors such as the route of application, age, sex and body weight of the subject, and accordingly, the dosage does not limit the scope of the present invention in any way.

As used herein, the term “hair” refers to any hair that grows on an individual (including, for example, a mammal). For example, in the present disclosure, hair may include at least one selected from the group consisting of head hair, eyebrows, and eyelashes, but is not limited thereto.

One aspect of the present disclosure provides a use of the above-described anhydrous composition for enhancing moisturization of the body.

One aspect of the present disclosure provides a use of the above-described anhydrous composition for preparing a composition for enhancing moisturization of the body.

In an embodiment, the body further includes at least one selected from the group consisting of hair (for example: scalp, eyelashes, and eyebrows), skin (for example: elbows and cuticles), nails, and toenails, but is not limited thereto.

In an embodiment, the composition for enhancing moisturization of the body may be a cosmetic composition, a food composition, an oral composition, a non-therapeutic composition, a non-therapeutic oral composition, a pharmaceutical composition, or an external preparation composition.

Hereinafter, the present invention will be described in detail with reference to Examples for specifically describing the present invention. The following Examples are provided for illustrative purposes only to aid in the understanding of the present invention, and the scope and spirit of the present invention are not limited thereby.

### 1. Preparation of Anhydrous Composition

Hair oils of Examples 1 to 7 and Comparative Examples 1 to 5 were prepared according to the compositions shown in the following Table 1. Specifically, the emollient, fatty alcohol, wax and/or poorly soluble substance were mixed with a base and stirred to prepare the oil. The bases of Examples 1 to 7 and Comparative Examples 2 to 5 include dimethicone, dimethiconol, and oil, and the base of Comparative Example 1 includes a low-viscosity silicone oil (linear dimethicone (0.65 cs to 100 cs)) and oil.

TABLE 1

| | | | | | | | | | Com- | Com- | Com- | Com- | Com- |
|---|---|---|---|---|---|---|---|---|---|---|---|---|---|
| | | | | | | | | | par- | par- | par- | par- | par- |
| | | | | | | | | | ative | ative | ative | ative | ative |
| | | Exam- | Exam- | Exam- | Exam- | Exam- | Exam- | Exam- | Exam- | Exam- | Exam- | Exam- | Exam- |
| Component (wt %) | | ple 1 | ple 2 | ple 3 | ple 4 | ple 5 | ple 6 | ple 7 | ple 1 | ple 2 | ple 3 | ple 4 | ple 5 |
| Emol- | Cetyl | 5 | 1 | 10 | — | — | 5 | 5 | 5 | 5 | 5 | 5 | — |
| lient | ethylhexanoate | | | | | | | | | | | | |
| | Caprylic/ | — | — | — | 5 | — | — | — | — | — | — | — | — |
| | Capric | | | | | | | | | | | | |
| | triglyceride | | | | | | | | | | | | |
| | Hydrogenated | — | — | — | — | 5 | — | — | — | — | — | — | — |
| | polyisobutene | | | | | | | | | | | | |
| Fatty | Myristyl | — | — | — | — | — | 1 | — | — | — | — | — | — |
| alcohol | alcohol (C14) | | | | | | | | | | | | |
| | Behenyl | 1 | 0.1 | 5 | 1 | 1 | — | 1 | 1 | 1 | 1 | — | 1 |
| | alcohol (C22) | | | | | | | | | | | | |
| Wax | Jojoba ester | 1 | 0.1 | 5 | 1 | 1 | 1 | — | 1 | 1 | — | 1 | 1 |
| | (melting point | | | | | | | | | | | | |
| | 70° C.) | | | | | | | | | | | | |
| | Hydrogenated | — | — | — | — | — | — | 1 | — | — | — | — | — |
| | vegetable oil | | | | | | | | | | | | |
| | (melting point | | | | | | | | | | | | |
| | 25° C.) | | | | | | | | | | | | |
| Poorly | Ceramide NP | 0.5 | 0.1 | 1 | 0.5 | 0.5 | 0.5 | 0.5 | 0.5 | — | 0.5 | 0.5 | 0.5 |
| soluble | | | | | | | | | | | | | |
| substance | | | | | | | | | | | | | |

| | | | | | | | | | | | | |
|---|---|---|---|---|---|---|---|---|---|---|---|---|
| Base | to 100 | to 100 | to 100 | to 100 | to 100 | to 100 | to 100 | to 100 | to 100 | to 100 | to 100 | to 100 |
| Viscosity (cSt) | 1000 | 500 | 10,000 | 1000 | 1000 | 1000 | 1000 | 100 | 1000 | 1000 | 1000 | 1000 |

Viscosities in the Examples and the Comparative Examples were measured using product test method No. 213/30. Specifically, 100 g of each of the Examples and Comparative Examples was placed in a viscosity bottle, and then the viscosity of the content was measured using a rotational viscometer with a No. 3 spindle at 30° C. and 12 rpm for 2 minutes. When the viscosity was 10,000 or more, a No. 4 spindle was used.

### 2. Evaluation of Dissolution and Stability of Poorly Soluble Substance in Formulation

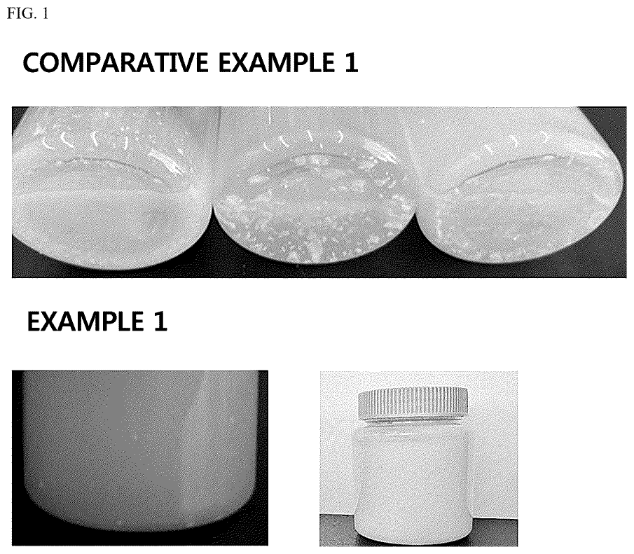

It was confirmed by the naked eye whether the poorly soluble substances were effectively dissolved and dispersed in the compositions of the Examples and the Comparative Examples. Specifically, the compositions of the Examples and the Comparative Examples were stored in a constant temperature/humidity chamber at 30° C. for 24 hours. As a result, in Comparative Example 1, in which the viscosity measured at 30° C. was less than 300 cSt, the poorly soluble substance was settled without being dissolved properly. Meanwhile, in the Example having a viscosity of 300 cSt or more measured at 30° C., the poorly soluble substance was not settled, but was uniformly distributed in the formulation (see FIG. 1).

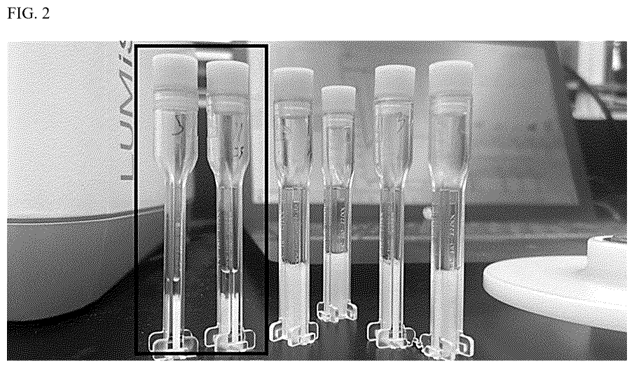

Further, the stabilities of the Examples and the Comparative Examples were analyzed using a LumiSizer, which is an apparatus for analyzing the dispersion stability and particle size distribution of a material under centrifugal force. First, the temperature and humidity environment stabilization time was secured, such that a controlled environment with a set temperature of 40° C. and a humidity of 50% in the apparatus could be reached. After the apparatus was set under conditions of a temperature of 40° C. and a humidity of 50%, the physical stability of the formulation was measured based on a 3-hour cycle. As a result of the analysis, the Example having a viscosity of 300 cSt or more measured at 30° C. exhibited excellent dispersion stability without any observation of phase separation or precipitation (FIG. 2, four test tubes from the right). Meanwhile, in the Comparative Examples in which some of the components for stabilizing poorly soluble substances were excluded (Comparative Example 3: Wax excluded, Comparative Example 4: Fatty alcohol excluded), it was confirmed that the upper and lower layers were separated and the dispersion stability was poor (the two test tubes from the left in FIG. 2). FIG. 2 is a photograph of samples after LumiSizer analysis, illustrating, from left to right, Comparative Example 3, Comparative Example 4, Example 4, Example 5, Example 6, and Example 7.

### 3. Evaluation of Feeling of Use and Moisturizing Effect Durability

### (1) Evaluation of Feeling of Use

A panel consisting of 20 female subjects aged 20 to 26 years old evaluated the feeling of use in Examples 1 to 7 and Comparative Examples 2 to 5.

The feeling of use was evaluated after using each of the Examples and the Comparative Examples once a day for two weeks. The evaluation was performed using a 9-point scale (the higher the score, the better), and specifically, for ‘hair softness,’ subjects were given a higher score when the hair felt soft upon use and the feeling of use was good, and for ‘residual feeling on the hands upon use,’ subjects were given a higher score when the residual feeling on the hands upon use was lighter and more refreshing. Comparative Example 1 was excluded from the evaluation because the poorly soluble substance was precipitated without being dissolved well, or the stability of the formulation was poor.

The average evaluation scores of the feeling of use of the 20 subjects are shown in the following Table 2, and in the feeling of use evaluation results in Table 2, X indicates poor quality (1 to 3 points), A indicates fair quality (4 to 6 points), and O indicates excellent quality (7 to 9 points).

### (2) Evaluation of Moisturizing Effect Durability

The moisturizing effect durability of Examples 1 to 7 and Comparative Examples 2 to 5 was evaluated.

Specifically, after shampooing, the contents were pumped twice onto towel-dried tresses and evenly applied to measure the moisture content. Among 120 bundles of black hair tresses in total (dyed once) of the tresses used in the test, 60 bundles tested for the hair moisture sustainability (infrared moisture meter FD-660) and 60 bundles tested for the hair moisture sustainability (Epsilon E100) were used to check the moisturizing power before use, after one use, 8 hours after use, and 24 hours after use.

As a result, Examples 1 to 7, in which ceramide was stably contained in the formulation, had a hair moisturizing durability effect. Meanwhile, Comparative Example 2, which did not include ceramide, had no hair moisturizing effect. When the moisture content increased to 10% or less 24 hours after use compared to immediately after use, it was marked with an X, and when the moisture content increased by 10% or more, it was marked with an O. In Example 1, the moisture content increased by 42% 24 hours after use compared to immediately after use.

TABLE 2

| | | | | | | | | Comparative | Comparative | Comparative | Comparative |
|---|---|---|---|---|---|---|---|---|---|---|---|
| Quality Evaluation | Exam- | Exam- | Exam- | Exam- | Exam- | Exam- | Exam- | Example | Example | Example | Example |
| Criteria | ple 1 | ple 2 | ple 3 | ple 4 | ple 5 | ple 6 | ple 7 | 2 | 3 | 4 | 5 |
| Feeling of use | ◯ | ◯ | ◯ | ◯ | ◯ | Δ | ◯ | ◯ | ◯ | X | X |
| (softness of hair) | | | | | | | | | | | |
| Felling of use | ◯ | ◯ | ◯ | ◯ | ◯ | ◯ | Δ | ◯ | X | ◯ | ◯ |
| (residual feeling | | | | | | | | | | | |
| on the hands) | | | | | | | | | | | |
| Moisturizing effect | ◯ | ◯ | ◯ | ◯ | ◯ | ◯ | ◯ | X | ◯ | ◯ | ◯ |
| durability | | | | | | | | | | | |

As shown in Table 2, Comparative Examples 3 to 5, which did not contain either one of the fatty alcohol, emollient, or wax, showed poor results such as an unfavorable feeling of use such as a residual feeling or sticky feeling on the hands upon use, or poor hair softness after use. Comparative Example 2, which did not include ceramide, a poorly soluble substance, did not have a good moisturizing effect on hair. Examples 1 to 7 may provide excellent moisturizing effects on hair by stably dissolving/dispersing poorly soluble substances, and simultaneously exhibit excellent feeling of use and softness when applied to hair.

### 4. Evaluation of Residual Amount of Ceramide in Hair for Each Formulation

The residual amounts of ceramide in hair were compared and evaluated between a high-viscosity oil formulation and a low-viscosity emulsion formulation, which included ceramide.

First, a low-viscosity oil formulation and a high-viscosity oil formulation according to one aspect of the present disclosure, which included ceramide, were prepared. Each formulation was prepared so as to include the same amount of ceramide. More specifically, a common cream-type hair mist formulation was selected as the low-viscosity emulsion formulation, and more specifically, an O/W type emulsion formulation including a cationic surfactant and a higher alcohol (1-docosanol; a saturated fatty alcohol including 22 carbon atoms) was used. The low-viscosity emulsion formulation had a low ratio of surfactant and higher alcohol, and thus, is a formulation having a viscosity of less than 300 cSt (2 min, 12 rpm, No. 4 spindle). The high-viscosity oil formulation was used in Example 1 in Table 1 above.

Each of the formulations prepared above (the low-viscosity emulsion formulation and the high-viscosity oil formulation) was subjected to three hair tresses. Treatment conditions such as the amount of formulation applied, washing time, and amount of shampoo were controlled to be the same. To prepare standard and test solutions for evaluating the residual amount of ceramide, ceramide was precisely weighed and dissolved in methane to prepare a standard stock solution at a concentration of 1 mg/mL. The test solution was precisely weighed to be 0.1 g for an untreated hair tress and 0.05 g for a hair tress treated with each formulation (the low-viscosity emulsion formulation and the high-viscosity oil formulation), each was adjusted to 10 mL by adding methanol, and subjected to ultrasonic extraction for 1 hour.

To prepare a hair cross-section sample, the hair tress sample was cut into a size of 2 cm and placed in a mold containing O.C.T compound (Tissue-TeK) such that no air bubbles were generated between the hairs, and then the hair was fixed using liquid nitrogen. Thereafter, the sample was sectioned into 5 μm- or 10 μm-thick sections using a cryotome (CM1850-7-1) to evaluate the residual amount of ceramide. Thereafter, the residual amount of ceramide was calculated using MS Condition and UPLC.

As a result of the analysis, a group to which the high-viscosity oil formulation was applied (test group) had about 1.63-fold higher residual ceramide levels in the hair after seven cumulative treatments compared to a group to which the low-viscosity emulsion formulation was applied (control).

These results indicate that the high-viscosity oil formulation according to one aspect of the present disclosure is more effective in delivering and retaining ceramide in hair, which is consistent with the results confirmed in ‘3. Evaluation of feeling of use and moisturizing effect durability’ above.

### 5. Evaluation of Effects of Ceramide Penetration into Hair

When the composition of the high-viscosity oil formulation according to one aspect of the present disclosure is applied to hair, it was evaluated whether ceramide effectively penetrated the hair.

First, a high-viscosity oil formulation including ceramide was prepared. Specifically, Example 1 in Table 1 above was used as the high-viscosity oil formulation.

A untreated white hair tress (control) and a white hair tress treated with Example 1 (test group) were prepared, then cut into a size of 2 cm, and placed in a mold containing O.C.T compound (Tissue-TeK) to fix the hair. Thereafter, the hair cross-section was analyzed by sectioning the sample into 10 μm-thick sections using a cryotome (CM1850-7-1).

After mosaicking the image using an objective lens microscope, real-time Raman spectra were obtained by specifying points. Ceramide peaks were identified through Raman spectroscopy, extracted, and visualized by chemical image processing.

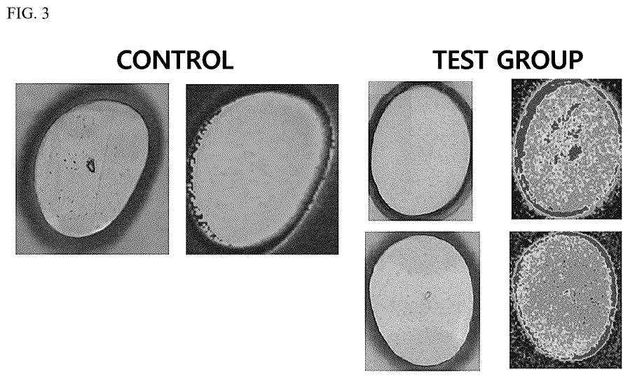

As a result of the analysis, penetration of ceramide into the cuticle layer of the hair was clearly observed in the test group to which the example composition was applied, compared to the untreated group (right photograph in FIG. 3; ceramide is shown in red).

These results indicate that the high-viscosity oil formulation according to one aspect of the present disclosure is more effective in delivering and retaining ceramide in hair, which is consistent with the results confirmed in ‘3. Evaluation of feeling of use and moisturizing effect durability’ above.

1. An anhydrous composition comprising: a fatty alcohol; an emollient; a wax; and a poorly soluble substance, and having a viscosity of 300 cSt or more at 30° C.

2. The anhydrous composition of claim 1, wherein the fatty alcohol is a C14-22 saturated fatty alcohol.

3. The anhydrous composition of claim 1, wherein the fatty alcohol is at least one selected from the group consisting of myristyl alcohol, cetyl alcohol, stearyl alcohol, and behenyl alcohol.

4. The anhydrous composition of claim 1, wherein the emollient is at least one selected from the group consisting of an ester-based emollient, a triglyceride-based emollient, and a hydrocarbon-based emollient.

5. The anhydrous composition of claim 4, wherein the ester-based emollient is an ester of a C3-C14 carboxylic acid and a C3-C20 alcohol.

6. The anhydrous composition of claim 4, wherein the ester-based emollient is at least one selected from the group consisting of cetyl ethylhexanoate, a C12-15 alkyl benzoate, and isopropyl myristate.

7. The anhydrous composition of claim 4, wherein the triglyceride-based emollient is an ester of a C8-C10 carboxylic acid and glycerol.

8. The anhydrous composition of claim 4, wherein the triglyceride-based emollient is at least one selected from the group consisting of caprylic/capric triglyceride, camellia oil, and Abyssinian oil.

9. The anhydrous composition of claim 4, wherein the hydrocarbon-based emollient is at least one selected from the group consisting of hydrogenated polyisobutene, squalane, squalene, and polybutene.

10. The anhydrous composition of claim 1, wherein the wax is natural wax.

11. The anhydrous composition of claim 1, wherein the wax has a melting point of 25° C. to 75° C.

12. The anhydrous composition of claim 1, wherein the wax is at least one selected from the group consisting of jojoba esters, MANGIFERA INDICA (MANGO) seed butter, ASTROCARYUM MURUMURU seed butter, and hydrogenated vegetable oil.

13. The anhydrous composition of claim 1, wherein the poorly soluble substance is any one or more selected from the group consisting of ceramide, madecassoside, hyaluronic acid, retinol, retinoid, coenzyme Q-10, ursolic acid, oleanolic acid, lipoic acid, proteins, and water-soluble substances.

14. The anhydrous composition of claim 13, wherein the ceramide is at least one selected from the group consisting of ceramide NS, ceramide AS, ceramide EOS, ceramide NDS, ceramide ADS, ceramide EODS, ceramide NP, ceramide AP, ceramide EOP, ceramide NH, ceramide AH, and ceramide EOH.

15. The anhydrous composition of claim 1, further comprising at least one of dimethicone, dimethiconol, cyclomethicone, and oil.

16. The anhydrous composition of claim 1, wherein the fatty alcohol, emollient, and wax form particles.

17. The anhydrous composition of claim 1, wherein the anhydrous composition does not comprise a surfactant.

18. The anhydrous composition of claim 1, wherein the anhydrous composition is for hair.

19. The anhydrous composition of claim 1, wherein the anhydrous composition is an anhydrous cosmetic composition.
